(19) United States
(12) Patent Application Publication
(10) Pub. No.: US 2006/0131762 A1
(43) Pub. Date: Jun. 22, 2006

(54) Method for metal-organic production of organic intermediate products by means of aryl lithium-bases

(76) Inventors: Andreas Meudt, Sulzbach (DE); Bernd Lehnemann, Sulzbach (DE); Michael Erbes, Sulzbach (DE); Klaus Forstinger, Sulzbach (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/526,237

(22) PCT Filed: Aug. 21, 2003
(86) PCT No.: PCT/EP03/09252

§ 371 Date: Feb. 28, 2005

(30) Foreign Application Priority Data
Aug. 31, 2002 (DE) 102 40 262.0

(51) Int. Cl. C07D 333/22; C07D 213/63; C07F 1/02

(52) U.S. Cl. 260/665 R; 546/290; 549/76; 548/530; 549/475

(57) ABSTRACT

The invention relates to a method for the production of substituted aromatic compounds by producing lithium arylene and by reacting it with suitable electrophiles. The method comprises the following steps (step 1); an aryl lithium compound ( auxiliary base”) is initially produced by reacting a halogen aromatic compound with lithium metal; said compound is subsequently (step 2) reacted for deprotonation of the aromatic substrate in order to form the corresponding lithium aromatic compound which is subsequently (step 3) reacted with a corresponding electrophile to form the desired substituted aromatic compound, see page 2 of the description.

The invention relates to a process for preparing substituted aromatic compounds by generation of lithium aryls and reaction of these with suitable electrophiles, in which a haloaromatic is firstly (step 1) reacted with lithium metal to generate an aryllithium compound (“auxiliary base”) which is subsequently (step 2) reacted with the aromatic substrate to deprotonate the latter and form the corresponding lithioaromatic, and this is finally (step 3) reacted with an appropriate electrophile to form the desired substituted aromatic compound (equation 1).
Step 1: Generation of the Base 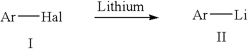
Step 2: Deprotonation of the Substrate 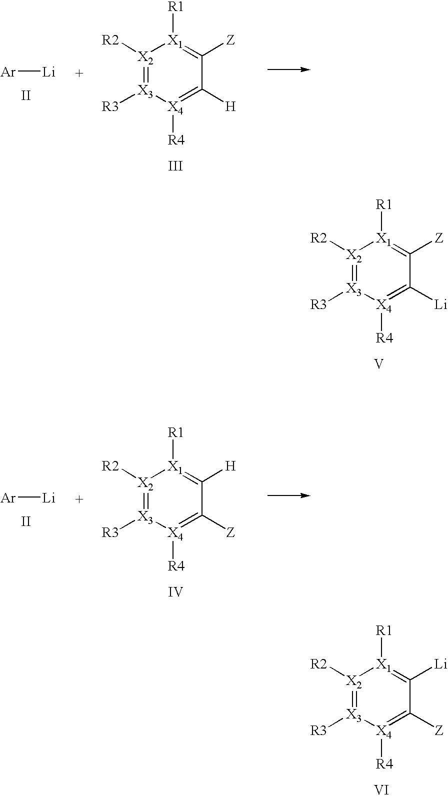
Step 3: Reaction of the Aryllithium Compound with an Electrophile 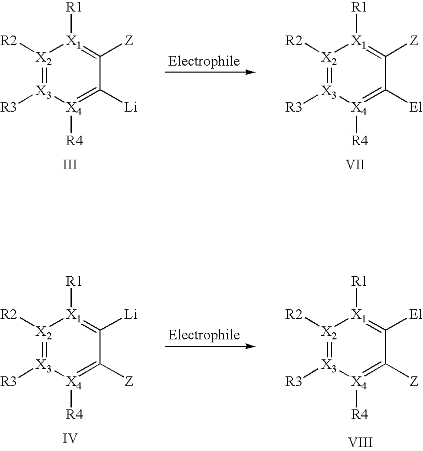
(Equation 1)

The upswing in organometallic chemistry, in particular that of the element lithium, in the preparation of compounds for the pharmaceutical and agrochemicals industries and also for numerous further applications has progressed almost exponentially in the past few years if the number of applications or the quantity of products produced in this way is plotted against a time axis. Significant reasons for this are, firstly, the evermore complex structures of the fine chemicals required for the pharmaceuticals and agrochemicals sectors and, secondly, the virtually unlimited synthetic potential of organolithium compounds for building up complex organic structures. Almost any organblithium compound can be generated easily by means of the modern Arsenal or organometallic chemistry and be reacted with virtually any electrophiles to form the respective desired product. Most organolithium compounds are generated using of the following routes:

(1) the most important route is without doubt halogen-metal exchange in which (usually) bromoaromatics are reacted with n-butyllithium at low temperatures

(2) some organolithium compounds can be prepared by reacting bromoaromatics with lithium metal, as long as no interfering groups which react with Li metal are present in the molecule,

(3) furthermore, the deprotonation of organic compounds using lithium alkyls (e.g. BuLi) or lithium amides (e.g. LDA or LiNSi) is very important

It can be seen from this that the use of commercial alkyllithium compounds is required for the major part of this chemistry, with N-BuLi mostly being used here. The synthesis n-BuLi and related lithioaliphatics is technically complicated and requires a great deal of know-how, as a result of which n-butyllithium, s-butyllithium, tert-butyllithium and similar molecules are, from an industrial viewpoint, offered at very high prices. This is the most important but far from the only disadvantage of this otherwise very advantageous and widely usable reagent.

Owing to the extreme sensitivity and, in concentrated solutions, pyrophoric nature of such lithioaliphatics, very costly logistics systems for transport, introduction into the metering reservoir and metering have to be provided for the large amounts (annular production quantities of from 5 to 500 metric tons) wanted in large-scale industrial production.

Furthermore, the reactions of n-, s- and tert-butyllithium form either butanes (deprotonations), butyl halides (halogen-metal exchange, one equivalent of BuLi) or butene and butane (halogen-metal exchange) which are gaseous at room temperature and are given off in the necessary hydrolytic work-ups of the reaction mixture. As a result, complicated offgas purifications or appropriate incineration facilities are also necessary in order to meet strict pollution laws. As a way of avoiding this, specialist companies are offering alternatives such as n-hexyllithium which do not result in formation of butanes, but are significantly more expensive than butyllithium.

A further disadvantage is that complex solvent mixtures are obtained after the work-up. Adding to the high reactivity of alkyllithium compounds toward ethers, which are virtually always used as solvents for the subsequent reactions, alkyllithium compounds can usually not be marketed in these solvents. Although the producers offer a broad range of alkyllithium compounds in various concentrations in various hydrocarbons, halogen-metal exchange reactions, for example, do not proceed in pure hydrocarbons, so that one is forced to work in mixtures of ethers and hydrocarbons. After hydrolysis, this results in water-containing mixtures of ethers and hydrocarbons whose separation is complicated and can in many cases not be carried out economically at all. However, recycling of the solvents used is an indispensable prerequisite for large-scale industrial production.

For the reasons mentioned, it would therefore be very desirable to have a process in which the use of such commercially available organolithium compounds can be avoided. An in-situ variant using alkyl halides/lithium metal have been described by us in the German patent application 101 50 615.4-44, which is not a prior publication. For various reasons associated with the easier isolation of the protonated auxiliary base, with a frequently smaller critical by-product spectrum and with often cheaper aryl halides, the use of aryl halides/lithium metal as “auxiliary bases” would in many cases be even more advantageous.

The present invention achieves these objects and provides a process for preparing aryllithium compounds of the formulae (V) and (VI) and reacting them with suitable electrophiles to give compounds of the formulae (VII) and (VIII), in which aryl halides of the formula (I) are firstly (“two-stage variant”) or if appropriate in the presence of the electrophilic compound (substrate) (“in-situ variant”) with lithium metal to generate a lithium compound (II), this is used for deprotonating the aromatic substrate (III) or (IV), and is finely converted by addition of the electrophilic component into the target compound of the formula (VII) or (VIII) (equation 1),
Step 1: Generation of the Base 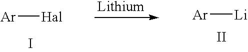
Step 2: Deprotonation of the Substrate 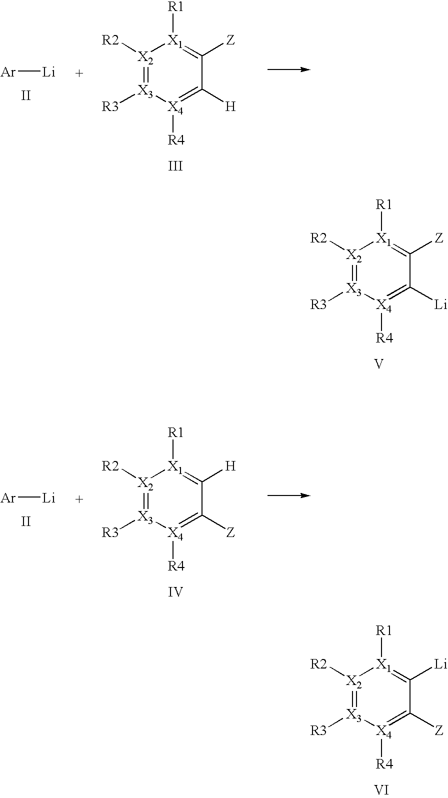
Step 3: Reaction of the Aryllithium Compound with an Electrophile 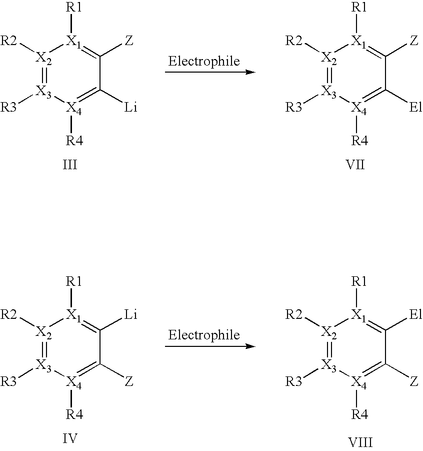
(Equation 1)
where Ar is phenyl, alkyl-substituted phenyl, fluorine- or chlorine-substituted phenyl, naphthyl-, alkyl-substituted naphthyl or is biphenyl,
Hal=fluorine, chlorine, bromine or iodine,
the radicals X₁₋₄ are, independently of one another, either carbon, XᵢRᵢ (i=1-4) can symbolize nitrogen, or two radicals XᵢRᵢ which are adjacent or connected via a formal double bond can together be O (furans), S (thiophenes), NH or NRᵢ (pyrroles),
Z is, in the case of benzoidal aromatics, a group which activates the ortho position, for example CF₃, OCF₃, Cl, F, Oalkyl, Oaryl, Salkyl, Saryl, CH₂OH, CH₂OR, CH(OR)₂, CONR₂, NHR, NR₂, or in the case of heterocycles has the same meaning as R₁₋₄,
the radicals R₁₋₄ are substituents selected from the group consisting of hydrogen, methyl, primary, secondary or tertiary, cyclic or acyclic alkyl radicals having from 2 to 12 carbon atoms, substituted cyclic or acyclic alkyl groups, alkoxy, dialkylamino, alkylamino, arylamino, diarylamino, phenyl, substituted phenyl, alkylthio, diarylphosphino, dialkylphosphino, dialkylaminocarbonyl or diarylaminocarbonyl, monoalkylaminocarbonyl or monoarylaminocarbonyl, CO₂alkyl, CO₂⁻, 1-hydroxyalkyl, 1-alkoxyalkyl, fluorine or chlorine, CN or heteroaryl, where two adjacent radicals R₁₋₄ can together correspond to a fused-on aromatic or aliphatic ring,
and “Electrophile” is any electrophilic component which can be reacted with aryllithium compounds.

Preferred compounds of the formula (III) or (IV) which can be reacted by the process of the invention are, for example, benzenes, furans, thiophenes, pyridines, pyridazines, pyrimidines, pyrazines, N-substituted pyrroles, benzofurans, indoles or naphthalenes, to name only a few.

The organolithium compounds prepared in this way can be reacted with any electrophilic compound by methods of the prior art. C,C couplings, for example, can be carried out by reaction with carbon electrophiles, boronic acids can be prepared by reaction with boron compounds, and an efficient route to organosilanes is provided by reaction with halosilanes or alkoxysilanes.

As haloaromatics, it is possible to use all available or procurable fluoroaromatics, chloroaromatics, bromoaromatics or iodoaromatics, since lithium metal reacts readily with all haloaromatics in ether solvents, giving quantitative yields in virtually all cases. Preference is given to using chloroaromatics or bromoaromatics, since iodo compounds are often expensive and fluorine compounds lead to the formation of LiF which can, as HF, lead to material problems in the later aqueous work-ups. In specific cases, however, such halides may be able to be used advantageously. Particular preference is naturally given to commercially available aryl chlorides such as chlorobenzene, but especially the isomeric chlorotoluenes. It is even possible to use industrial isomer mixtures which in many cases are even cheaper.

The reaction is carried out in a suitable organic solvent, preferably an ether solvent such as tetrahydrofuran (THF), dioxane, diethyl ether, di-n-butyl ether, a glyme or diisopropyl ether. Preference is given to using THF.

Adding to the high reactivity of aryllithium compounds, in particular also toward the ethers used as solvents, the preferred reaction temperatures are in the range from −100 to +35° C., particularly preferably from −70 to +25° C.

Apart from the abovementioned advantages of the procedure according to the invention (costs, logistics, safety, gaseous by-products), a further advantage is that it is possible to work at quite high concentrations of organolithium compounds. This results, in particular, from the fact that the use of, for example, butyl lithium in commercial concentrations introduces from five to six times the volume of solvent. Preference is given to concentrations of the aliphatic or aromatic intermediates of the formula (II) or (IV) of from 5 to 30% by weight, in particular from 12 to 25% by weight.

In addition, high selectivities are frequently also observed, which is due to the fact that the reaction can be carried out in pure ethers instead of ether/hydrocarbon mixtures.

In the preferred embodiment, haloaromatic and aromatic substrates are added simultaneously or as a mixture to the lithium metal in the ether. In this one-pot variant, the lithioaromatic is formed first and then immediately deprotonates the actual substrate. In a second preferred embodiment which can be employed especially when the aromatic can undergo secondary reactions with metallic lithium, it is possible firstly to generate the aryllithium compound in an ether by reaction of the haloaromatic and lithium and only then add the aromatic substrate before the targeted molecule is finally produced by reaction with an electrophile.

In addition, it has surprisingly been found that in the preferred embodiment as a one-pot reaction, significantly higher yields than when ArLi is generated first and the aromatic substrate is only added subsequently are observed in very many cases.

In the present process, the lithium can be used as dispersion, powder, turnings, sand, granules, pieces, bars or in another form, with the size of the lithium particles not being relevant to quality but merely influencing the reaction times. Preference is therefore given to relatively small particle sizes, for example granules, powders or dispersions. The amount of lithium added per mole of halogen to be reacted is from 1.95 to 2.5 mol, preferably from 1.98 to 2.15 mol.

In all cases, significant increases in the reaction rates and frequently also increases in yield can be observed when organic redox systems, for example biphenyl, 4,4′-di-tert-butylbiphenyl or anthracene, are added. It is usually sufficient to add these compounds in amounts of <0.5 mol %, usually even <0.02 mol %.

Aromatics which can be used for the deprotonation are all compounds which are sufficiently acidic to be able to be deprotonated under the conditions according to the invention. These are firstly all those aromatics which have strongly “ortho-directing” substituents Z, i.e., in particular, aromatics which bear alkoxy, CF₃, F, Cl, substituted amino, CN, heteroaryl, aminoalkyl, hydroxyalkyl or similar radicals. The mode of action of such radicals is based on the substituents making it possible for the lithium ions to coordinate to the aliphatic base, as a result of which the counterion Ar can then be deprotonated very easily in the ortho position.

Furthermore, all heterocycles which are strongly acidic due to the combination of a plurality of effects, for example furan, may be mentioned in this context. Here, the protons are sufficiently acidic to make deprotonation possible as a result of, inter alia, the inductive effect of the oxygen and also the sp hybridization and the angular stress on the a carbon. The same applies to other heterocycles.

The lithioaromatics generated according to the invention can be reacted with electrophilic compounds by methods with which those skilled in the art are familiar, with carbon, boron and silicon electrophiles being of particular interest with a view to the intermediates required for the pharmaceutical and agrochemicals industries.

The carbon electrophiles come, in particular, from one of the following categories (the products as shown in brackets in each case):

oxirane, substituted oxiranes (ArCH₂CH₂OH, ArCR₂CR₂OH) where R═R¹ (identical or different)

azomethines (ArCR₂—NR′H)

aryl or alkyl cyanates (benzonitriles)

nitroenolates (oximes)

immonium salts (aromatic amines)

haloaromatics, aryl triflates, other aryl sulfonates (biaryls)

carbon dioxide (ArCOOH)

carbon monoxide (Ar—CO—CO—Ar)

aldehydes, ketones (ArCHR¹—OH, ArCR₂—OH)

α,β-unsaturated aldehydes/ketones (ArCH(OH)-vinyl, CR¹(OH-vinyl ketenes (ARC(═O)CH₃ in the case of ketene, (ArC(═O)—R¹ in the case of substituted ketenes)

alkali metal and alkaline earth-metal salts of carboxylic acids (ArCHO in the case of formates, ArCOCH₃ in the case of acetates, ArR¹CO in the case of R¹COOMet)

aliphatic nitriles (ArCOCH₃ in the case of acetonitrile, ArR¹ CO in the case of R¹CN)

aromatic nitriles (ArCOAr′)

amides (ArCHO in the case of HCONR₂, ArC(═O)R in the case of RCONR′₂)

esters (Ar₂C(OH)R¹) or

alkylating agents such as alkyl halides or alkyl sulfonates (Ar-alkyl).

Boron electrophiles used are compounds of the formula BW₃, where the radicals W are identical or different and are each C₁-C₆-alkoxy, fluorine, chlorine, bromine, iodine, N(C₁-C₆-alkyl)₂ or S(C₁-C₅-alkyl), with preference being given to trialkoxyboranes, BF₃ OR₂, BF₃ THF, BCl₃ or BBr₃, particularly preferably trialkoxyboranes.

Silicon electrophiles used are compounds of the formula SiW₄, where the radicals W are identical or different and each C₁-C₆-alkoxy, fluorine, chlorine, bromine, iodine, N(C₁-C₆-alkyl)₂ or S(C₁-C₅-alkyl), with preference being given to tetralkoxysilanes, tetrachlorosilanes or substituted alkylhalosilanes or arylhalosilanes or substituted alkylalkoxysilanes or arylalkoxysilanes.

The work-ups are generally aqueous, with either water or aqueous mineral acids being added or the reaction mixture being introduced into water or aqueous mineral acids. To achieve the best yields, the pH of the product to be isolated is in each case such, i.e. usually a slightly acidic pH, or in the case of heterocycles also a slightly alkaline pH. The reaction products are obtained, for example, by extraction and evaporation of the organic phases, or, as an alternative, the organic solvents can also be distilled off from the hydrolysis mixture and the product which then precipitates can be isolated by filtration.

The purities of the products from the process of the invention are generally high, but a further purification step, for example by recrystallization with addition of small amounts of activated carbon, may be necessary for special applications (pharmaceutical precursors). The yields of the reaction products are from 70 to 99%; typical yields are, in particular, from 85 to 95%.

The process of the invention provides a very economical method of bringing about the transformation of an aromatic hydrocarbon into any radicals in a very economical way.

The process of the invention is illustrated by the following examples, without the invention being restricted thereto.

### EXAMPLE 1

### Preparation of 5-formylfuran-2-boronic acid from furfural diethyl acetal and p-chlorotoluene

A mixture of 22.3 g of p-chlorotoluene (0.176 mol), 27.2 g of furfural diethyl acetal (0.16 mol) is added dropwise to a suspension of 2.35 g of lithium granules (0.34 mol) and 0.02 g of biphenyl in 300 g of THF at −65° C. over a period of one hour, with 1.5 hours being selected as metering time. When the conversion of the p-chlorotoluene as determined by GC is >97% (total time of 9 h), 18.3 g of trimethyl borate (0.176 mol) are added dropwise at the same temperature over a period of 30 minutes. After stirring for another 30 minutes at −65° C., the reaction mixture is added to 120 g of water, 37% strength HCl is added to adjust the pH to 6.3 and THF and toluene are distilled off at up to 35° C. under reduced pressure. The pH is then adjusted to 1.5, the mixture is stirred until the product has precipitated completely and the product is filtered off. After washing with a little cold water and a little cold acetone, drying gives 18.2 g of 5-formyl-2-furanboronic acid (0.130 mol, 81.5%) in the form of a fine, beige powder; HPLC purity: >99.8% a/a.

### EXAMPLE 2

### Preparation of 2,6-dimethoxyphenylboronic acid from resorcinol dimethyl ether and chlorotoluene (isomer mixture)

A mixture of 22.3 g of technical-grade chlorotoluene (isomer mixture, 0.176 mol) and 22.1 g of resorcinol dimethyl ether (0.16 mol) is added to a suspension of 2.35 g of lithium-granules (0.345 mol) and 0.02 g of biphenyl in 220 g of THF at 0° C. over a period of 2 hours. When the conversion of the chlorotoluene as determined by GC is >99% (total time of 6 h), the reaction mixture is cooled to −50° C. 16.6 g of trimethyl borate (0.16 mol) are subsequently added dropwise over a period of 30 minutes. After stirring for another 30 minutes at −50° C., the reaction mixture is added to 120 g of water, 37% strength HCl is added to adjust the pH to 6.3 and THF and toluene are distilled off at 35° C. under reduced pressure. 25 ml of methylcyclohexane are added to the product suspension, the colorless product is filtered off with suction and washed once with 25 ml of cold methylcyclohexane and once with 25 ml of cold water. Drying gives 27.5 g of 2,6-dimethoxyphenylboronic acid (0.151 mol, 94%, melting point: 107° C.) in the form of colorless crystals; HPLC purity: >99% a/a.

### EXAMPLE 3

### Preparation of 2-trifluoromethyl-6-chlorobenzoic acid

22.3 g of p-chlorotoluene (0.176 mol) dissolved in 100 ml of THF are added dropwise to a suspension of 2.35 g of lithium granules (0.34 mol) in 150 g of THF at −30° C. over a period of 1 hour. When the conversion as determined by GC has reached at least 99% (total time of 5 h), the mixture is cooled to −60° C. and the metered addition of 3-chlorobenzotrifluoride (31.0 g, 0.172 mol) is subsequently commenced (30 min). After stirring for another 1 hour, the introduction of anhydrous carbon dioxide is commenced. After CO₂ absorption has ceased, the mixture is stirred at −60° C. for another 30 minutes. The reaction mixture is subsequently added to 100 g of water, 37% strength HCl is added to adjust the pH to 3.4 and the solvents are distilled off at up to 55° C. under reduced pressure. The colorless product is filtered off with suction and dried to give 2-trifluoromethyl-6-chlorobenzoic acid (yield: 69%) in the form of colorless crystals; HPLC purity: >99% a/a. Further 2-trifluoromethyl-6-chlorobenzoic acid can be obtained by extraction of the mother liquor with dichloromethane, drying over sodium sulfate and evaporation; total yield: 89%.

### EXAMPLE 4

### Preparation of 2-trifluoromethyl-6-chlorobenzaldehyde

22.3 g of p-chlorotoluene (0.176 mol) dissolved in 100 ml of THF are added dropwise to a suspension of 2.35 g of lithium granules (0.34 mol) in 150 g of THF at −30° C. over a period of 1 hour. When the conversion as determined by GC has reached at least 99% (total time of 5 h), the mixture is cooled to −70° C. and the metered addition of 3-chlorobenzotrifluoride (31.0 g, 0.172 mol) is subsequently commenced (30 min). After stirring for another 1 hour, the metered addition of methyl formate is commenced (0.2 mol, 12.0 g). After stirring for another 30 minutes, the reaction mixture is subsequently added to 100 g of water, 37% strength HCl is added to adjust the pH to 6.5 and the solvents are distilled off at up to 45° C. under reduced pressure. The residue is extracted twice with dichloromethane, the solution is dried over sodium sulfate and evaporated to dryness to leave 2-trifluoromethyl-6-chlorobenzaldehyde as a slightly yellowish oil; HPLC purity: >97%, yield 94%.

### EXAMPLE 5

### Preparation of 2,6-difluoroacetophenone from 1,3-difluorobenzene and acetic anhydride

A solution of phenyllithium in THF is firstly produced by reacting 65.2 g of chlorobenzene with 7.0 g of lithium granules in 400 g of THF at −25° C. When a conversion of >98% (GC a/a) has been reached, the mixture is cooled to −65° C. and 1,3-difluorobenzene (55 g) is then added over a period of 30 minutes. After stirring for another 30 minutes, the resulting solution of 2,6-difluoro-1-lithiobenzene is added dropwise to a solution of 88 g of acetic anhydride in 250 g of THF which has been cooled to −5° C. After the usual aqueous work-up, 2,6-difluoroacetophenone is obtained in a yield of 88%.

### EXAMPLE 6

### Preparation of 2-furylboronic acid from furan and triisopropyl borate (“in-situ variant”)

p-Chlorotoluene (0.25 mol) and furan (0.24 mol) are simultaneously added dropwise from two dropping funnels to a suspension of 0.52 mol of lithium granules in 300 g of THF at −15° C. over a period of 30 minutes. After a conversion of the chlorotoluene of >97% has been reached (7 h), the mixture is cooled to −60° C. and trimethyl borate (0.275 mol) is subsequently added dropwise as quickly as possible (temperature must not rise above −55° C., since otherwise too much of the corresponding boric acid is formed). The mixture is finally stirred for another 15 minutes and thawed to room temperature. After addition to 450 g of water, adjustment of the pH to 6.5 by means of dilute hydrochloric acid and vacuum distillation of the organic solvents (THF, toluene) under very mild conditions, the mixture is cooled to 5° C. Filtration of the resulting suspension, washing with ice water and cooled THF/water 80:20 and drying at max. 35° C./50 mbar gives 2-furanboronic acid in a yield of 86%.

1. A process for preparing compounds of the formulae (VII) and (VIII) via aryllithium compounds of the formulae (V) and (VI) and reaction of these with suitable electrophiles, aryl halides of the formula (I) are reacted with lithium metal to generate a lithium compound (II), this is used for deprotonating the aromatic substrate (III) or (IV), and is finally converted by addition of the electrophilic component into the target compound of the formula (VII) or (VIII) (equation 1), and the steps 1 to 3 are carried out as a one-pot reaction,
Step 1: Generation of the Base 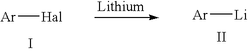
Step 2: Deprotonation of the Substrate 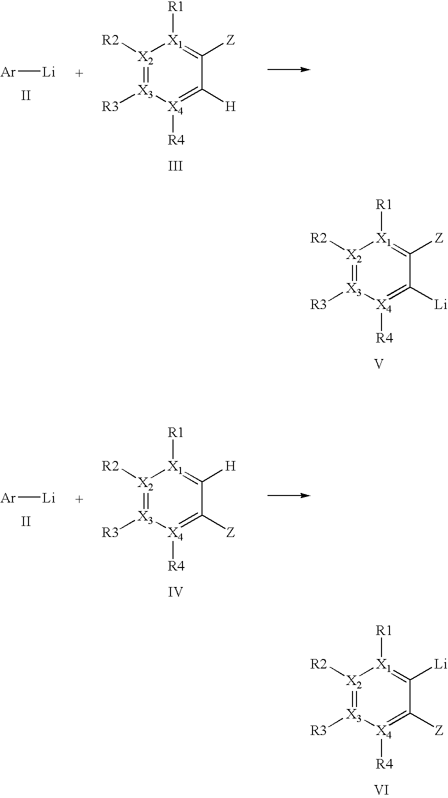
Step 3: Reaction of the Aryllithium Compound with an Electrophile 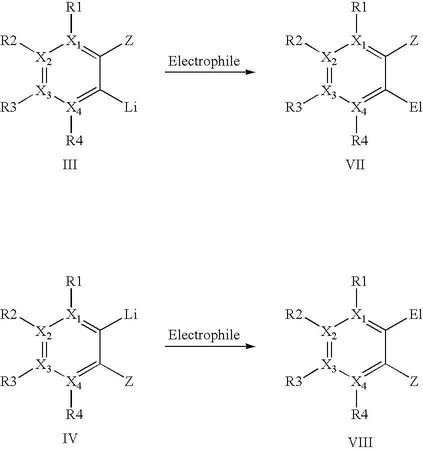
(Equation 1)
where Ar is phenyl, alkyl-substituted phenyl, fluorine- or chlorine-substituted phenyl, naphthyl-, alkyl-substituted naphthyl or is biphenyl, Hal is a halogen selected from the group consisting of fluorine, chlorine, bromine, iodine, and mixtures thereof,
the radicals X₁₋₄ are, independently of one another, either carbon, XᵢRᵢ (i=1-4) can symbolize nitrogen, or two radicals XᵢRᵢ which are adjacent or connected via a formal double bond can together be O (furans), S (thiophenes), NH or NRᵢ (pyrroles),
Z is, in the case of benzoidal aromatics, a group which activates the ortho position, for example CF₃, OCF₃, Cl, F, Oalkyl, Oaryl, Salkyl, Saryl, CH₂OH, CH₂OR, CH(OR)₂, CONR₂, NHR, NR₂, or in the case of heterocycles has the same meaning as R₁₋₄,
the radicals R₁₄ are substituents selected from the group consisting of hydrogen, methyl, primary, secondary or tertiary, cyclic or acyclic alkyl radicals having from 2 to 12 carbon atoms, substituted cyclic or acyclic alkyl groups, alkoxy, dialkylamino, alkylamino, arylamino, diarylamino, phenyl, substituted phenyl, alkylthio, diarylphosphino, dialkylphosphino, dialkylaminocarbonyl or diarylaminocarbonyl, monoalkylaminocarbonyl or monoarylaminocarbonyl, CO₂alkyl, CO₂⁻, 1-hydroxyalkyl, 1-alkoxyalkyl, fluorine or chlorine, CN or heteroaryl, where two adjacent radicals R₁₋₄ can together correspond to a fused-on aromatic or aliphatic ring,
and wherein the electrophile is any electrophilic component which can be reacted with aryllithium compounds.

2. The process as claimed in claim 1, wherein the compounds of the formula (III) or (IV) are selected from the group consisting of benzenes, furans, thiophenes, pyridines, pyridazines, pyrimidines, pyrazines, N-substituted pyrroles, benzofurans, indoles, naphthalenes, and mixtures thereof.

3. The process as claimed in claim 1, characterized in that the electrophile is a compound selected from the group consisting of oxirane, substituted oxirane, azomethine, aryl or alkyl cyanate, nitroenolate, immonium salts, haloaromatics, aryl triflates, other aryl sulfonates, carbon dioxide, carbon monoxide, aldehydes, ketones, α,β-unsaturated aldehydes or ketones, ketenes, alkali metal or alkaline earth metal salts of carboxylic acids, aliphatic nitriles, aromatic nitriles, amides, esters and alkylating agents and boron electrophiles of the formula BW₃, where the radicals W are identical or different and are each C₁-C₆-alkoxy, fluorine, chlorine, bromine, iodine, N(C₁-C₆-alkyl)₂ or S(C₁-C₅-alkyl), and silicon electrophiles of the formula SiW₄, where the radicals W are identical or different and are each C₁-C₆-alkoxy, fluorine, chlorine, bromine, iodine, N(C₁-C₆-alkyl)₂ or S(C₁-C₅-alkyl), and mixtures thereof.

4. The process of claim 1, characterized in that the reaction is carried out in an organic ether solvent.

5. The process of claim 1, wherein said process is carried out at a reaction temperature in a range from −100 to +35° C.

6. The process of claim 1, wherein the aromatic intermediates of the formula (II) or (IV) are present in a concentration in a range from 5 to 30% by weight.

7. The process of claim 1, wherein the lithium metal is added in an amount per mole of halogen reacted ranging from 1.95 to 2.5 mol.

8. The process of claim 1, further comprising adding organic redox systems to the one pot reaction.

9. The process of claim 8, wherein an amount of said organic redox system added is less than 0.5 mol-%.
